(19) United States
(12) Patent Application Publication
(10) Pub. No.: US 2021/0236593 A1
(43) Pub. Date: Aug. 5, 2021

(54) METHODS OF SUPPORTING WEIGHT MAINTENANCE BY DECREASING THE ACTIVITY OF FAM46A

(71) Applicant: SOCIETE DES PRODUITS NESTLE S.A., Vevey (CH)

(72) Inventors: Jerome Carayol, Pully (CH); Armand Valesia, Chavannes-pres-Renens (CH); Jorg Hager, Houtaud (FR)

(21) Appl. No.: 17/196,592

(22) Filed: Mar. 9, 2021

Related U.S. Application Data
(62) Division of application No. 16/462,706, filed on May 21, 2019.

(30) Foreign Application Priority Data
Dec. 1, 2016 (EP) 16201666.1

(51) Int. Cl. A61K 38/16; C12N 15/113; G01N 33/50

(52) U.S. Cl. CPC A61K 38/16; C12N 15/113; G01N 2333/575; G01N 33/5044; C12N 2310/14; G01N 33/5023

(57) ABSTRACT

An agent capable of decreasing the activity of FAM46A for use in supporting weight maintenance and/or treating obesity.

### CROSS REFERENCE TO RELATED APPLICATIONS

The present application is a continuation of U.S. patent application Ser. No. 16/462,706 filed May 21, 2019, which is a National Stage of International Application No. PCT/EP2017/080737, filed on Nov. 29, 2017, which claims priority to European Patent Application No. 16201666.1, filed on Dec. 1, 2016, the entire contents of which are being incorporated herein by reference.

### FIELD OF THE INVENTION

The present invention relates to agents which are capable of modulating the activity of FAM46A and the use of such agents in therapy, in particular in supporting weight maintenance and treating obesity. The invention also relates to methods of identifying such agents.

### BACKGROUND TO THE INVENTION

Obesity is a chronic metabolic disorder that has reached epidemic proportions in many areas of the world. Obesity is the major risk factor for serious co-morbidities such as type 2 diabetes mellitus, cardiovascular disease, dyslipidaemia and certain types of cancer (World Health Organ. Tech. Rep. Ser. (2000) 894: i-xii, 1-253).

Obesity refers to a condition in which an individual weighs more than usual as a result of excessive accumulation of energy from carbohydrate, fat and the like. The additional weight is typically retained in the form of fat under the skin or around the viscera.

Empirical data suggests that a weight loss of at least 10% of the initial weight results in a considerable decrease in the risk of obesity related co-morbidities (World Health Organ. Tech. Rep. Ser. (2000) 894: i-xii, 1-253). However, the capacity to lose weight shows large inter-subject variability.

Obesity is induced when the amount of energy intake exceeds the amount of energy consumed. Thus, in order to ameliorate obesity, a method of decreasing the amount of energy intake from fat, carbohydrate and the like or a method of increasing the amount of energy consumption by promoting in vivo metabolism is desired. Accordingly, improvements in dietary habit and exercise are considered to be effective methods for the prevention and amelioration of obesity and obesity-related disorders. For example, it has long been recognised that low calorie diet (LCD) interventions can be very efficient in reducing weight and that this weight loss is generally accompanied by an improvement in the risk of obesity related co-morbidities, in particular type 2 diabetes mellitus (World Health Organ. Tech. Rep. Ser. (2000) 894: i-xii, 1-253).

Although a number of methods are known for promoting weight loss, subjects face the risk of regaining lost weight once a period of weight loss intervention has been completed. Such regression risks reducing or potentially completely reversing any benefits that were associated with the loss of weight.

Accordingly, there remains a significant need not only for improved methods of promoting weight loss, but also for methods for supporting weight maintenance (preventing or reducing the regain of lost weight, and hence supporting maintenance of weight at a level similar to that achieved following weight loss intervention). Such improvements would provide more complete treatments for obesity, thus decreasing the risk of obesity-related disorders.

Obesity is associated with a number of physiological changes in the body including differences in the levels of certain proteins which are either higher or lower in obese subjects than in individuals with a normal body weight (Singla, Bardoloi and Parkash, World J Diabetes (2010)). Moreover, it has been shown that the blood levels for many of these proteins changes dramatically during a weight loss intervention (Van Dijk et al. Plos One (2010)). One such example is the protein leptin, a satiety associated hormone, the levels of which are increased in obese subjects and then which decreases during a weight loss intervention.

However, little is known if these changes in protein levels are causally associated with obesity and weight loss or if they are just a reflection of the obese status and the weight loss intervention.

One way to explore a possible causality for changes in protein levels is to study if the levels of these proteins are controlled genetically i.e. if there are genetic variants that are associated with different levels of these proteins. Analyses that study such relationships are referred to as gene-protein-quantitative trait studies. Using modern molecular biology techniques these can be carried out on a very large scale involving hundreds of thousands of genetic variants and testing their association with the levels of hundreds to thousands of proteins. A genetic variant (so called single nucleotide polymorphisms, short SNP's) that is associated with the levels of a specific protein are referred to protein quantitative trait loci (pQTL's).

A pQTL can be close to the gene coding for the protein that shows the level changes and is called a cis-pQTL or it can be associated with a different genomic region (trans-pQTL). Trans-pQTL's are often genetic variants linked to proteins that in their turn regulate the levels of the protein of interest. Thus pQTL studies allow to identify new regulators of proteins associated with traits of interest like for example obesity and weight loss.

### SUMMARY OF THE INVENTION

The inventors carried out an analysis of protein quantitative trait loci (pQTL) on weight loss intervention data obtained from the Diogenes study. This study is a pan-European, randomised and controlled dietary intervention study investigating the effects of dietary protein and glycaemic index on weight loss and weight maintenance in obese and overweight families in eight European centres (Larsen et al. (2009) Obesity Rev. 11: 76-91). In brief, the Diogenes study subjected overweight/obese participants to a low-calorie diet (LCD) weight loss phase (CID1), in which subjects followed an 8 week ModifastR diet (approximately 800 kCal/day), followed by a 6 months weight maintenance phase (CID2).

In the present context, the pQTL are genomic loci that contribute to variations in protein levels during the LCD weight loss phase. The inventors specifically analysed pQTL associated with proteins which exhibited expression changes that correlated with weight loss.

The inventors observed differential expression of leptin levels in blood during the LCD phase that was significantly associated with weight loss, a finding which correlates with the understanding in the field that leptin is linked to appetite suppression and control. The inventors also observed a pQTL associated with leptin levels in blood in a region of chromosome 6 between the branched-chain alpha-keto acid dehydrogenase E1 B subunit (BCKDHB) gene and the FAM46A gene.

To distinguish, which of the two genes might be responsible for the pQTL signal, the inventors investigated genetic variants in the pQTL region which were associated with the gene expression levels of either gene. On further investigation, the inventors observed that an genetic variants in the region were associated with gene expression levels for FAM46A only, indicating that this gene/protein is the more likely to be functionally linked with the leptin pQTL FAM46A inversely correlated with leptin levels i.e. lower levels of FAM46A were associated with higher leptin levels.

In addition, the inventors observed that FAM46A is highly correlated with BCL2-associated athanogene 6 (BAG6) in the data from the Diogenes study. In support of this finding, body-mass index (BMI) is known to be the strongest determinant of BAG6 splicing in subcutaneous fat. The cellular stress-related protein BAG6 is known to play roles in gene and protein regulation, and apoptosis. In particular, BAG6 is a central regulator of the cellular content of HSP70, a further cellular stress-related protein that is correlated to leptin in type 2 diabetes and also in the Diogenes data. While not wishing to be bound by theory, the correlation between FAM46A and the cellular stress-related proteins BAG6 and HSP70 indicate a further link between FAM46A and the success of a dietary intervention during which cells are subjected to increased stress.

Accordingly, the inventors have established two independent links between FAM46A and weight control and appetite suppression, in particular during a dietary intervention period, thus providing for new interventions to support weight maintenance and the treatment of obesity.

Accordingly, in one aspect the invention provides an agent capable of decreasing the activity of FAM46A for use in supporting weight maintenance and/or treating or preventing obesity.

In another aspect, the invention provides an agent capable of decreasing the activity of FAM46A for use in suppressing the appetite of a subject. In another aspect, the invention provides an agent capable of decreasing the activity of FAM46A for use in prolonging satiety. In another aspect, the invention provides an agent capable of decreasing the activity of FAM46A for use in reducing food intake by a subject. In another aspect, the invention provides an agent capable of decreasing the activity of FAM46A for use in reducing fat deposition in a subject.

In another aspect, the invention provides the use of an agent capable of decreasing the activity of FAM46A for supporting weight maintenance. In another aspect, the invention provides the use of an agent capable of decreasing the activity of FAM46A for suppressing the appetite of a subject. In another aspect, the invention provides the use of an agent capable of decreasing the activity of FAM46A for prolonging satiety. In another aspect, the invention provides the use of an agent capable of decreasing the activity of FAM46A for reducing food intake by a subject. In another aspect, the invention provides the use of an agent capable of decreasing the activity of FAM46A for reducing fat deposition in a subject.

In another aspect, the invention provides a method of supporting weight maintenance comprising administering an agent of the invention to a subject in need thereof. In another aspect, the invention provides a method of suppressing the appetite of a subject comprising administering an agent of the invention to a subject in need thereof. In another aspect, the invention provides a method of prolonging satiety comprising administering an agent of the invention to a subject in need thereof. In another aspect, the invention provides a method of reducing food intake by a subject comprising administering an agent of the invention to a subject in need thereof. In another aspect, the invention provides a method of reducing fat deposition in a subject comprising administering an agent of the invention to a subject in need thereof. In another aspect, the invention provides a method of treating or preventing obesity comprising administering an agent of the invention to a subject in need thereof.

The activity of FAM46A may be decreased in comparison with the activity in the absence of the agent of the invention. The activity of FAM46A may be decreased by, for example, at least 1%, 2%, 3%, 4%, 5%, 6%, 7%, 8%, 9%, 10%, 15%, 20%, 25%, 50%, 75% or 100%.

The agent may be, for example, a FAM46A antagonist or inhibitor, or the agent may decrease the level of FAM46A in a cell.

In one embodiment, the agent suppresses the appetite of a subject.

In one embodiment, the agent increases or prolongs satiety. In another embodiment, the agent reduces food intake by a subject. In another embodiment, the agent reduces fat deposition in a subject.

In one embodiment, the agent is administered to a subject during or after a weight loss intervention. In a preferred embodiment, the agent is administered to a subject during a weight loss intervention. The weight loss intervention may be, for example, a diet regimen (e.g. a low-calorie diet) and/or an exercise regimen.

In one embodiment, the agent increases leptin levels in a subject. The level of leptin may be increased in comparison with the level in the absence of the agent of the invention. The level of leptin may be increased by, for example, at least 1%, 2%, 3%, 4%, 5%, 6%, 7%, 8%, 9%, 10%, 15%, 20%, 25%, 50%, 75%, 100% or more.

In one embodiment, the agent decreases the level of FAM46A in a subject. In this context, “level” refers to the amount of FAM46A and may be measured, for example, by analysing the amount of protein expressed and/or by analysing the amount of the corresponding mRNA present. Preferably, the agent decreases the expression of FAM46A. For example, siRNAs, shRNAs, miRNAs or antisense RNAs may reduce expression of FAM46A.

In one embodiment, siRNAs may reduce expression of FAM46A.

The level of FAM46A may be decreased in comparison with the level in the absence of the agent of the invention. The level of FAM46A may be decreased by, for example, at least 1%, 2%, 3%, 4%, 5%, 6%, 7%, 8%, 9%, 10%, 15%, 20%, 25%, 50%, 75% or 100%.

In one embodiment, the agent is selected from the agents listed in Table 1.

In a preferred embodiment, the agent is selected from the group consisting of an siRNA, shRNA, miRNA, antisense RNA, polynucleotide, polypeptide or small molecule. The polypeptide may be, for example, an antibody. Thus, the agent of the invention may be in the form of a polynucleotide encoding an siRNA, shRNA, miRNA or antisense RNA that targets FAM46A, or a polypeptide (e.g. an antibody). The polynucleotide may be in the form of a vector, such as a viral vector.

The agent of the invention may be an agent identified by a method of the invention.

In another aspect, the invention provides a method of identifying an agent capable of supporting weight maintenance and/or treating or preventing obesity in a subject comprising the steps:
(a) contacting a preparation comprising a FAM46A polypeptide or polynucleotide with a candidate agent; and
(b) detecting whether the candidate agent affects the activity of the FAM46A polypeptide or polynucleotide.

The effect on activity of the FAM46A polypeptide or polynucleotide may be analysed by comparing the activities of the FAM46A polypeptide or polynucleotide in the presence and absence (i.e. a control experiment) of the candidate agent.

In another aspect, the invention provides a method of identifying an agent that decreases the activity of FAM46A comprising the steps:
(a) contacting a preparation comprising a FAM46A polypeptide or polynucleotide with a candidate agent; and
(b) detecting whether the candidate agent affects the activity of the FAM46A polypeptide or polynucleotide.

The methods of the invention may be methods for identifying an agent capable of suppressing the appetite of a subject, increasing or prolonging satiety, reducing food intake by a subject and/or reducing fat deposition in a subject.

In one embodiment, the preparation comprising the FAM46A polypeptide or polynucleotide comprises a cell comprising the FAM46A polypeptide or polynucleotide.

In a preferred embodiment, the cell is an adipocyte.

In one embodiment, the method is for identifying an agent that decreases the expression of FAM46A.

In one embodiment, the method comprises detecting whether the candidate agent affects the levels of leptin produced by the cell.

In one embodiment the method comprises determining agents which bind to FAM46A followed by determining which of these agents are capable of modulating leptin levels.

In one embodiment, the candidate agent is a natural product.

In another aspect, the invention provides the use of FAM46A, or a polynucleotide encoding the same, in a method of identifying an agent that supports weight maintenance, suppresses the appetite of a subject, increases or prolongs satiety, reduces food intake by a subject, reduces fat deposition in a subject, and/or treats or prevents obesity.

In another aspect, the invention provides the use of an agent capable of decreasing the activity of FAM46A for manufacturing a medicament for use in supporting weight maintenance, suppressing the appetite of a subject, increasing or prolonging satiety, reducing food intake by a subject, reducing fat deposition in a subject, and/or treating or preventing obesity.

In another aspect, the invention provides a method of identifying an agent that decreases the expression of FAM46A comprising the steps:
(a) contacting a cell, preferably a cell expressing the FAM46A, with a candidate agent; and
(b) detecting whether the candidate agent decreases the expression of the FAM46A.

### DESCRIPTION OF THE DRAWINGS

FIG. 1

Locus-specific plots for leptin associated pOTL SNPs located in between FAM46A and BCKDHB genes. Association plot produced by the LocusZoom tool for distal genotyped and imputed SNPs associated to leptin protein expression change during LCD. SNPs p-values are plotted after −log 10 transformation with scale on the y axis and colours reflect pairwise linkage disequilibrium with the most associated SNP in the region (purple dot) based on the 1000 genomes EUR data set.

FIG. 2

FAM46A, LEP, PPARg, CEBPa and ADIPOQ expression during adipocyte differentiation. Relative mRNA expression for FAM46A and LEP were measured every 4 days from day 0 of differentiation to full maturation at day 15. The mRNA expression was determined by quantitative real-time PCR (qRT-PCR).

FIG. 3

FAM46A gene silencing effect on leptin protein expression in SGBS adipocytes. Individual data points corresponding to FIGS. 3A and 3B. SGBS cells were transfected with negative control siRNA (siNEG) or FAM46A-specific siRNA (siFAM46A) and experiments were performed at day nine. Gene expression levels are expressed relative to TBP (TATA Binding Protein) expression. Results are given as the mean +/−SD (standard deviation) for n=9.

FIG. 4

Overexpression of FAM46A in SGBS adipocytes. SGBS cells were transfected with 0.5 μg full length FAM46A cDNA for 48h and resulted in a 20-fold increase of FAM46A expression. FAM46A mRNA was assessed by qRT/PCR at day nine. Gene expression levels are expressed relative to TBP (TATA Binding Protein) expression. The data are shown as the means+/−SD (n=3) with *P<0.05, **P<0.01 for statistical significance calculated using the Student's t test (p_{Basal}=3.0×10−4, p_{Insulin}=1.0×10−4).

FIG. 5

FAM46A overexpression resulted in a significant 28% and 24% (p=0.034 and 0.038, one-sided t test, for both basal and insulin stimulated states, respectively) reduction of insulin and non-insulin stimulated leptin release, respectively

### DETAILED DESCRIPTION OF THE INVENTION

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

FAM46A

FAM46A (also known as HBV X-transactivated gene 11 protein; HBV XAg-transactivated protein 11; C6orf37; and XTP11) has been observed to be strongly expressed in placental tissue, the pineal gland and the pituitary gland. The first exon of the FAM46A gene harbours a variable number tandem repeat that has been linked with certain retinal diseases and colon cancer.

In one embodiment, the FAM46A is human FAM46A.

An example amino acid sequence of the FAM46A is the sequence deposited under NCBI Accession No. NP_060103.2.

An example amino acid sequence of the FAM46A is:

(SEQ ID NO: 1)
MAEGEGYFAMSEDELACSPYIPLGGDFGGGDFGGGDFGGGDFGGGGSFG
GHCLDYCESPTAHCNVLNWEQVQRLDGILSETIPIHGRGNFPTLELQPS
LIVKVVRRRLAEKRIGVRDVRLNGSAASHVLHQDSGLGYKDLDLIFCAD
LRGEGEFQTVKDVVLDCLLDFLPEGVNKEKITPLTLKEAYVQKMVKVCN
DSDRWSLISLSNNSGKNVELKFVDSLRRQFEFSVDSFQIKLDSLLLFYE
CSENPMTETFHPTIIGESVYGDFQEAFDHLCNKIIATRNPEEIRGGGLL
KYCNLLVRGFRPASDEIKTLQRYMCSRFFIDFSDIGEQQRKLESYLQNH
FVGLEDRKYEYLMTLHGVVNESTVCLMGHERRQTLNLITMLAIRVLADQ
NVIPNVANVTCYYQPAPYVADANFSNYYIAQVQPVFTCQQQTYSTWLPC
N

An example nucleotide sequence encoding the FAM46A is the sequence deposited under NCBI Accession No. NM_017633.2.

An example nucleotide sequence encoding the FAM46A is:

(SEQ ID NO: 2)
ATGGCGGAGGGTGAAGGGTACTTCGCCATGTCTGAGGACGAGCTGGCCT
GCAGCCCCTACATCCCCCTAGGCGGCGACTTCGGCGGCGGCGACTTCGG
CGGCGGCGACTTCGGCGGCGGCGACTTCGGCGGTGGCGGCAGCTTCGGT
GGGCATTGCTTGGACTATTGCGAAAGCCCTACGGCGCACTGCAATGTGC
TGAACTGGGAGCAAGTGCAGCGGCTGGACGGCATCCTGAGCGAGACCAT
TCCGATTCACGGGCGCGGCAACTTCCCCACGCTCGAGCTGCAGCCGAGC
CTGATCGTGAAGGTGGTGCGGCGGCGCCTGGCCGAGAAGCGCATTGGCG
TCCGCGACGTGCGCCTCAACGGCTCGGCAGCCAGCCATGTCCTGCACCA
GGACAGCGGCCTGGGCTACAAGGACCTGGACCTCATCTTCTGCGCCGAC
CTGCGCGGGGAAGGGGAGTTTCAGACTGTGAAGGACGTCGTGCTGGACT
GCCTGTTGGACTTCTTACCCGAGGGGGTGAACAAAGAGAAGATCACACC
ACTCACGCTCAAGGAAGCTTATGTGCAGAAAATGGTTAAAGTGTGCAAT
GACTCTGACCGATGGAGTCTTATATCCCTGTCAAACAACAGTGGCAAAA
ATGTGGAACTGAAATTTGTGGATTCCCTCCGGAGGCAGTTTGAATTCAG
TGTAGATTCTTTTCAAATCAAATTAGACTCTCTTCTGCTCTTTTATGAA
TGTTCAGAGAACCCAATGACTGAGACATTTCACCCCACAATAATCGGGG
AGAGCGTCTATGGCGATTTCCAGGAAGCCTTTGATCACCTTTGTAACAA
GATCATTGCCACCAGGAACCCAGAGGAAATCCGAGGGGGAGGCCTGCTT
AAGTACTGCAACCTCTTGGTGAGGGGCTTTAGGCCCGCCTCTGATGAAA
TCAAGACCCTTCAAAGGTATATGTGTTCCAGGTTTTTCATCGACTTCTC
AGACATTGGAGAGCAGCAGAGAAAACTGGAGTCCTATTTGCAGAACCAC
TTTGTGGGATTGGAAGACCGCAAGTATGAGTATCTCATGACCCTTCATG
GAGTGGTAAATGAGAGCACAGTGTGCCTGATGGGACATGAAAGAAGACA
GACTTTAAACCTTATCACCATGCTGGCTATCCGGGTGTTAGCTGACCAA
AATGTCATTCCTAATGTGGCTAATGTCACTTGCTATTACCAGCCAGCCC
CCTATGTAGCAGATGCCAACTTTAGCAATTACTACATTGCACAGGTTCA
GCCAGTATTCACGTGCCAGCAACAGACCTACTCCACTTGGCTACCCTGC
AATTAA

A further example amino acid sequence of the FAM46A is the sequence deposited under NCBI Accession No. AAH07351.1.

A further example amino acid sequence of the FAM46A is:

(SEQ ID NO: 3)
MAEGEGYFAMSEDELACSPYIPLGGDFGGGDFGGGDFGGGDFGGGDFGG
GGSFGGHCLDYCESPTAHCNVLNWEQVQRLDGILSETIPIHGRGNFPTL
ELQPSLIVKVVRRRLAEKRIGVRDVRLNGSAASHVLHQDSGLGYKDLDL
IFCADLRGEGEFQTVKDVVLDCLLDFLPEGVNKEKITPLTLKEAYVQKM
VKVCNDSDRWSLISLSNNSGKNVELKFVDSLRRQFEFSVDSFQIKLDSL
LLFYECSENPMTETFHPTIIGESVYGDFQEAFDHLCNKIIATRNPEEIR
GGGLLKYCNLLVRGFRPASDEIKTLQRYMCSRFFIDFSDIGEQQRKLES
YLQNHFVGLEDRKYEYLMTLHGVVNESTVCLMGHERRQTLNLITMLAIR
VLADQNVIPNVANVTCYYQPAPYVADANFSNYYIAQVQPVFTCQQQTYS
TWLPCN

A further example nucleotide sequence encoding the FAM46A is the sequence deposited under NCBI Accession No. BC007351.2.

A further example nucleotide sequence encoding the FAM46A is:

(SEQ ID NO: 4)
ATGGCGGAGGGTGAAGGGTACTTCGCCATGTCTGAGGACGAGCTGGCCT
GCAGCCCCTACATCCCCCTAGGCGGCGACTTCGGCGGCGGCGACTTCGG
CGGCGGCGACTTCGGCGGCGGCGACTTCGGCGGCGGCGACTTCGGCGGT
GGCGGCAGCTTCGGTGGGCATTGCTTGGACTATTGCGAAAGCCCTACGG
CGCACTGCAATGTGCTGAACTGGGAGCAAGTGCAGCGGCTGGACGGCAT
CCTGAGCGAGACCATTCCGATTCACGGGCGCGGCAACTTCCCCACGCTC
GAGCTGCAGCCGAGCCTGATCGTGAAGGTGGTGCGGCGGCGCCTGGCCG
AGAAGCGCATTGGCGTCCGCGACGTGCGCCTCAACGGCTCGGCAGCCAG
CCATGTCCTGCACCAGGACAGCGGCCTGGGCTACAAGGACCTGGACCTC
ATCTTCTGCGCCGACCTGCGCGGGGAAGGGGAGTTTCAGACTGTGAAGG
ACGTCGTGCTGGACTGCCTGTTGGACTTCTTACCCGAGGGGGTGAACAA
AGAGAAGATCACACCACTCACGCTCAAGGAAGCTTATGTGCAGAAAATG
GTTAAAGTGTGCAATGACTCTGACCGATGGAGTCTTATATCCCTGTCAA
ACAACAGTGGCAAAAATGTGGAACTGAAATTTGTGGATTCCCTCCGGAG
GCAGTTTGAATTCAGTGTAGATTCTTTTCAAATCAAATTAGACTCTCTT
CTGCTCTTTTATGAATGTTCAGAGAACCCAATGACTGAGACATTTCACC
CCACAATAATCGGGGAGAGCGTCTATGGCGATTTCCAGGAAGCCTTTGA
TCACCTTTGTAACAAGATCATTGCCACCAGGAACCCAGAGGAAATCCGA
GGGGGAGGCCTGCTTAAGTACTGCAACCTCTTGGTGAGGGGCTTTAGGC
CCGCCTCTGATGAAATCAAGACCCTTCAAAGGTATATGTGTTCCAGGTT
TTTCATCGACTTCTCAGACATTGGAGAGCAGCAGAGAAAACTGGAGTCC
TATTTGCAGAACCACTTTGTGGGATTGGAAGACCGCAAGTATGAGTATC
TCATGACCCTTCATGGAGTGGTAAATGAGAGCACAGTGTGCCTGATGGG
ACATGAAAGAAGACAGACTTTAAACCTTATCACCATGCTGGCTATCCGG
GTGTTAGCTGACCAAAATGTCATTCCTAATGTGGCTAATGTCACTTGCT
ATTACCAGCCAGCCCCCTATGTAGCAGATGCCAACTTTAGCAATTACTA
CATTGCACAGGTTCAGCCAGTATTCACGTGCCAGCAACAGACCTACTCC
ACTTGGCTACCCTGCAATTAA

In one embodiment, the FAM46A comprises an amino acid sequence that has at least 70%, 80%, 85%, 90%, 95%, 96%, 97%, 98%, 99% or 100% identity to SEQ ID NO: 1 or 3, preferably wherein the amino acid sequence substantially retains the natural function of the protein represented by SEQ ID NO: 1 or 3.

In one embodiment, the FAM46A-encoding nucleotide sequence comprises a nucleotide sequence that has at least 70%, 80%, 85%, 90%, 95%, 96%, 97%, 98%, 99% or 100% identity to SEQ ID NO: 2 or 4, preferably wherein the protein encoded by the nucleotide sequence substantially retains the natural function of the protein represented by SEQ ID NO: 1 or 3.

In one embodiment, the FAM46A-encoding nucleotide sequence comprises a nucleotide sequence that encodes an amino acid sequence that has at least 70%, 80%, 85%, 90%, 95%, 96%, 97%, 98%, 99% or 100% identity to SEQ ID NO: 1 or 3, preferably wherein the amino acid sequence substantially retains the natural function of the protein represented by SEQ ID NO: 1 or 3.

Leptin

Leptin is a hormone involved in the regulation of energy balance by inhibiting hunger. It is synthesised by adipose cells, as well as other cells in the body.

Leptin opposes the actions of the hormone ghrelin, which promotes hunger, with both hormones targeting receptors in the arcuate nucleus of the hypothalamus to regulate appetite. Decreased sensitivity to leptin is observed in obese subjects, which results in an inability to detect satiety.

In one embodiment, the leptin is human leptin.

An example amino acid sequence of the leptin is the sequence deposited under NCBI Accession No. NP_000221.1.

An example amino acid sequence of the leptin is:

(SEQ ID NO: 5)
MHWGTLCGFLWLWPYLFYVQAVPIQKVQDDTKTLIKTIVTRINDISHTQ
SVSSKQKVTGLDFIPGLHPILTLSKMDQTLAVYQQILTSMPSRNVIQIS
NDLENLRDLLHVLAFSKSCHLPWASGLETLDSLGGVLEASGYSTEVVAL
SRLQGSLQDMLWQLDLSPGC

An example nucleotide sequence encoding the leptin is the sequence deposited under NCBI Accession No. NM_000230.2.

An example nucleotide sequence encoding the leptin is:

(SEQ ID NO: 6)
ATGCATTGGGGAACCCTGTGCGGATTCTTGTGGCTTTGGCCCTATCTTTT
CTATGTCCAAGCTGTGCCCATCCAAAAAGTCCAAGATGACACCAAAACCC
TCATCAAGACAATTGTCACCAGGATCAATGACATTTCACACACGCAGTCA
GTCTCCTCCAAACAGAAAGTCACCGGTTTGGACTTCATTCCTGGGCTCCA
CCCCATCCTGACCTTATCCAAGATGGACCAGACACTGGCAGTCTACCAAC
AGATCCTCACCAGTATGCCTTCCAGAAACGTGATCCAAATATCCAACGAC
CTGGAGAACCTCCGGGATCTTCTTCACGTGCTGGCCTTCTCTAAGAGCTG
CCACTTGCCCTGGGCCAGTGGCCTGGAGACCTTGGACAGCCTGGGGGGTG
TCCTGGAAGCTTCAGGCTACTCCACAGAGGTGGTGGCCCTGAGCAGGCTG
CAGGGGTCTCTGCAGGACATGCTGTGGCAGCTGGACCTCAGCCCTGGGTG
CTGA

The leptin amino acid sequence may include secretory signal sequences. Such signal sequences may be cleaved during the secretion process, thus the protein may naturally exist in a mature form lacking the signal sequence. The skilled person is readily able to determine such signal sequences using appropriate bioinformatic and molecular biology techniques. For example, residues 1-21 of SEQ ID NO: 5 may act as a signal sequence.

Weight Loss and Weight Maintenance

The term “weight loss” as used herein may refer to a reduction in parameters such as weight (e.g. in kilograms), body mass index (kg/m2), waist-hip ratio (e.g. in centimetres), fat mass (e.g. in kilograms), hip circumference (e.g. in centimetres) or waist circumference (e.g. in centimetres).

Weight loss may be calculated by subtracting the value of one or more of the aforementioned parameters at the end of an intervention (e.g. a diet and/or exercise regimen) from the value of the parameter at the onset of the intervention.

The degree of weight loss may be expressed as a percent change of one of the aforementioned weight phenotype parameters (e.g. a percent change in a subject's body weight (e.g. in kilograms) or body mass index (kg/m²)). For example, a subject may lose at least 10% of their initial body weight, at least 8% of their initial body weight, or at least 5% of their initial body weight. By way of example only, a subject may lose between 5 and 10% of their initial body weight.

In one embodiment, a degree of weight loss of at least 10% of initial body weight results in a considerable decrease in the risk of obesity-related co-morbidities.

The term “weight maintenance” as used herein may refer to the maintenance in parameters such as weight (e.g. in kilograms), body mass index (kg/m²), waist-hip ratio (e.g. in centimetres) fat mass (e.g. in kilograms), hip circumference (e.g. in centimetres) or waist circumference (e.g. in centimetres). Weight maintenance may refer to, for example, maintaining weight lost following an intervention (e.g. a diet and/or exercise regimen).

The degree of weight maintenance may be calculated by determining the change in one or more of the afore-mentioned parameters over a period of time. The period of time may be, for example, at least 5, 10, 15, 20, 25, 30, 35, 40, 45 or 50 weeks.

Weight maintenance supported by the agents of the invention may result in, for example, a change (e.g. gain) of less than 10%, 9%, 8%, 7%, 6%, 5%, 4%, 3%, 2% or 1% in one or more of the afore-mentioned parameters over a period of time.

The degree of weight maintenance may be expressed as the weight regained during a period following attainment of weight loss, for example as a percentage of the weight lost during attainment of weight loss.

Weight maintenance supported by the agents of the invention may result through suppression of a subject's appetite following administration of the agent. The subject may therefore have a reduced appetite compared to the appetite in the absence of the agent of the invention.

Weight maintenance supported by the agents of the invention may result through control of a subject's appetite following administration of the agent. The subject may therefore maintain control over their appetite and therefore maintain their weight, for example following a period of weight loss intervention.

In particular, the agents of the invention may support weight maintenance through appetite suppression or control during and/or following a period of weight loss intervention (e.g. a diet or exercise regime).

In one aspect, the invention provides the non-therapeutic use of an agent of the invention to maintain a healthy body composition, for example after a period of weight loss.

Obesity

The term “overweight” as used herein is defined for an adult human as having a body mass index (BMI) between 25 and 30.

The term “body mass index” as used herein means the ratio of weight in kg divided by the height in metres, squared.

The term “obesity” as used herein refers to a condition in which the natural energy reserve, stored in the fatty tissue of animals, in particular humans and other mammals, is increased to a point where it is associated with certain health conditions or increased mortality. The term “obese” as used herein is defined for an adult human as having a BMI greater than 30.

The term “normal weight” as used herein is defined for an adult human as having a BMI of 18.5 to 25, whereas the term “underweight” as used herein may be defined as a BMI of less than 18.5.

Obesity is a chronic metabolic disorder that has reached epidemic proportions in many areas of the world and is the major risk factor for serious co-morbidities such as type 2 diabetes mellitus, cardiovascular disease, dyslipidaemia and certain types of cancer (World Health Organ. Tech. Rep. Ser. (2000) 894: i-xii, 1-253).

The term “obesity-related disorder” as used herein refers to any condition which an obese individual is at an increased risk of developing. Obesity-related disorders include diabetes (e.g. type 2 diabetes), stroke, high cholesterol, cardiovascular disease, insulin resistance, coronary heart disease, metabolic syndrome, hypertension and fatty liver.

Methods of Screening

The invention provides agents that are capable of decreasing the activity of FAM46A, and additionally provides methods for identifying such agents.

The agents of the invention may be identified by methods that provide either qualitative or quantitative results. Furthermore, such methods may be used to characterise as well as identify agents of the invention.

The candidate agents may be any agents of potential interest, for example peptides, polypeptides (e.g. antibodies), nucleic acids or small molecules. Preferably, the candidate agents are compounds or mixtures of potential therapeutic interest. Preferably, the candidate agents are of low toxicity for mammals, in particular humans. In some embodiments, the candidate agents may comprise nutritional agents and/or food ingredients, including naturally-occurring compounds or mixtures of compounds such as plant or animal extracts.

The candidate agents may form part of a library of agents, for example a library produced by combinatorial chemistry or a phage display library. In one embodiment, the candidate agents form part of a library of plant bioactive molecules.

FAM46A Activity

The ability of a candidate agent to reduce the activity of a protein, for example an enzyme, may be expressed in terms of an 1050 value. The 1050 is the concentration of an agent that is required to give rise to a 50% reduction in the activity of the protein (e.g. a 50% reduction in enzymatic activity). The calculation of 1050 values is well known in the art.

Preferably, the agents of the invention have an 1050 value for inhibition of FAM46A of less than 100 μM, more preferably less than 10 μM, for example less than 1 μM, less than 100 nM or less than 10 nM.

Techniques for measuring FAM46A activity may be applied to FAM46A that has been isolated from a cell. The FAM46A may have been expressed using recombinant techniques. Preferably, the FAM46A has been purified.

In one embodiment, FAM46A activity is measured by determining modulation in leptin levels.

FAM46A Binding

The invention also provides methods of identifying agents which are capable of binding to FAM46A and, alternatively or additionally, characterising such binding. For example, the method may allow measurement of absolute or relative binding affinity, and/or enthalpy and entropy of binding. Binding affinity may be expressed in terms of the equilibrium dissociation (K_{d}) or association (Kₐ) constant.

A number of assay techniques are known in the art for identifying binding between a candidate agent and a protein. The assay technique employed is preferably one which is amenable to automation and/or high throughput screening of candidate agents. The assay may be performed on a disposable solid support such as a microtitre plate, microbead, resin or similar.

For example, target FAM46A may be immobilised on a solid support, for example a microbead, resin, microtitre plate or array. Candidate agents may then be contacted with the immobilised target protein. Optionally, a wash procedure may be applied to remove weakly or non-specifically binding agents. Any agents binding to the target protein may then be detected and identified. To facilitate the detection of bound agents, the candidate agents may be labelled with a readily detectable marker. The marker may comprise, for example, a radio label, an enzyme label, an antibody label, a fluorescent label, a particulate (e.g. latex or gold) label or similar.

Alternatively, the above procedure may be reversed and the candidate agents may be immobilised and the target FAM46A may be contacted with said immobilised agents. Optionally, a wash procedure may be applied to remove weakly or non-specifically bound target protein. Any agents to which FAM46A binds may then be detected and identified. To facilitate the detection of binding, the FAM46A may be labelled with a readily detectable marker as described above.

In addition to the assays described above, other suitable assay techniques are known in the art. Examples of such techniques include radioassays, fluorescence assays, ELISA, fluorescence polarisation, fluorescence anisotropy, isothermal titration calorimetry (ITC), surface plasmon resonance (SPR) and the like. These assays may be applied to identify agents which bind to FAM46A. Indeed, platforms for the automation of many of these techniques are widely known in the art to facilitate high-throughput screening.

More than one assay techniques may be used to provide a detailed understanding of a candidate agent's binding to FAM46A. For example, assays which provide qualitative binding information may be used as a first step in the method, followed by further assays using different techniques to provide quantitative binding data and/or data on the effect on activity of the target protein.

The assay techniques described above may be adapted to perform competition binding studies. For example, these techniques are equally suitable to analyse the binding of a protein to substrate or cofactor in the presence of a candidate agent. It will therefore be possible to use the above techniques to screen and identify agents that modulate the binding between a protein and its substrate or cofactor, thus having an effect on the protein's activity.

Preferably, the agents of the invention will bind with high affinity. For example, the agents of the invention will bind to FAM46A with a K_{d} of less than 100 μM, more preferably less than 10 μM, for example less than 1 μM, less than 100 nM or less than 10 nM.

Binding affinity may be measured using standard techniques known in the art, e.g. surface plasmon resonance, ELISA and so on (for instance as described above), and may be quantified in terms of either dissociation (K_{d}) or association (Kₐ) constants.

Bioinformatics-based approaches, such as in silico structure-guided screening, may also be used to identify agents of the invention.

FAM46A Levels

The invention provides agents for decreasing FAM46A levels. Levels of FAM46A may be equated with levels of expression of the protein in a cell or organism. Protein levels may be analysed directly or indirectly, for example by analysis of levels of mRNA encoding the protein.

Methods for analysing the expression of FAM46A may be employed in the invention to screen the effect of a candidate agent on the protein's levels.

A number of techniques are known in the art for determining the expression level of a protein. These techniques may be applied to test the effect of candidate agents on the expression level of FAM46A. The technique employed is preferably one which is amenable to automation and/or high throughput screening of candidate agents.

For example, screens may be carried out using cells harbouring polynucleotides encoding FAM46A operably linked to a reporter moiety. The reporter moiety may be operably linked to endogenous FAM46A-encoding genes. Alternatively, exogenous copies of FAM46A operably linked to a reporter moiety may be inserted into a cell. In this embodiment, the cell may be engineered to be deficient for natural FAM46A expression. Suitable reporter moieties include fluorescent labels, for example fluorescent proteins such as green, yellow, cherry, cyan or orange fluorescent proteins.

The term “operably linked” as used herein means the components described are in a relationship permitting them to function in their intended manner.

Such cells may be contacted with candidate agents and the level of expression of FAM46A may be monitored by analysing the level of reporter moiety expression in the cell. Fluorescent reporter moieties may be analysed by a number of techniques known in the art, for example flow cytometry, fluorescence activated cell sorting (FACS) and fluorescence microscopy. Expression levels of FAM46A may be compared before and after contact with the candidate agent. Alternatively, expression levels of FAM46A may be compared between cells contacted with a candidate agent and control cells.

Other methods may be used for analysing the expression of proteins, for example FAM46A. Protein expression may be analysed directly. For example, expression may be quantitatively analysed using methods such as SDS-PAGE analysis with visualisation by Coomassie or silver staining. Alternatively, expression may be quantitatively analysed using Western blotting or enzyme-linked immunosorbent assays (ELISA) with antibody probes which bind the protein product. FAM46A labelled with reporter moieties, as described above, may also be used in these methods. Alternatively, protein expression may be analysed indirectly, for example by studying the amount of mRNA corresponding to the protein that is transcribed in a cell. This can be achieved using methods such as quantitative reverse transcription PCR and Northern blotting.

Similar techniques may also be used for the analysis of leptin protein expression.

Agents

The invention provides agents that are capable of decreasing the activity of FAM46A, and additionally provides methods for identifying such agents.

The agents of the invention may be, for example, peptides, polypeptides (e.g. antibodies), nucleic acids (e.g. siRNAs, shRNAs, miRNAs and antisense RNAs) or small molecules. Preferably, the agents are of low toxicity for mammals, in particular humans. In some embodiments, the agents may comprise nutritional agents and/or food ingredients, including naturally-occurring compounds or mixtures of compounds such as plant or animal extracts.

Example agents that affect the activity of FAM46A include the agents recited in Table 1.

TABLE 1
Agents that decrease the activity of FAM46A.

| | Chemical | | |
|---|---|---|---|
| Chemical Name | ID | CAS RN | Interaction Actions |
| Acetaminophen | D000082 | 103-90-2 | Affects expression |
| Antirheumatic | D018501 | | Decreases expression |
| Agents | | | |
| Benzo(a)pyrene | D001564 | 50-32-8 | Decreases expression |
| Carbamazepine | D002220 | 298-46-4 | Affects expression |
| Clofibrate | D002994 | 637-07-0 | Decreases expression |
| Cuprizone | D003471 | 370-81-0 | Decreases expression |
| Cyclosporine | D016572 | 59865- | Decreases expression |
| | | 13-3 | |
| Cytarabine | D003561 | 147-94-4 | Decreases expression |
| Dibutyl Phthalate | D003993 | 84-74-2 | Decreases expression |
| Diethyl maleate | C014476 | 141-05-9 | Decreases expression |
| Diethylnitrosamine | D004052 | 55-18-5 | Affects |
| | | | cotreatment\|decreases |
| | | | expression |
| Diuron | D004237 | 330-54-1 | Decreases expression |
| Entinostat | C118739 | | Decreases expression |
| Fipronil | C082360 | 120068- | Decreases expression |
| | | 37-3 | |
| Hydrogen Peroxide | D006861 | 7722-84- | Affects expression |
| | | 1 | |
| Hydrogen Peroxide | D006861 | 7722-84- | Decreases expression |
| | | 1 | |
| Ionomycin | D015759 | 56092- | Affects |
| | | 81-0 | cotreatment\|decreases |
| | | | expression |
| Leflunomide | C045463 | | Decreases expression |
| Methylmercuric | C004925 | 115-09-3 | Decreases expression |
| chloride | | | |
| Nefazodone | C051752 | 83366- | Decreases expression |
| | | 66-9 | |
| Perfluoro-n- | C101814 | 2058-94- | Decreases expression |
| undecanoic acid | | 8 | |
| Phenobarbital | D010634 | 50-06-6 | Affects |
| | | | cotreatment\|decreases |
| | | | expression |
| Phenobarbital | D010634 | 50-06-6 | Affects expression |
| Pirinixic acid | C006253 | 50892- | Decreases expression |
| | | 23-4 | |
| Tetrachloro- | D013749 | 1746-01- | Decreases expression |
| dibenzodioxin | | 6 | |
| Tetra- | D013755 | 16561- | Affects |
| decanoylphorbol | | 29-8 | cotreatment\|decreases |
| Acetate | | | expression |
| Tretinoin | D014212 | 302-79-4 | Decreases expression |
| Trichostatin A | C012589 | 58880- | Decreases expression |
| | | 19-6 | |
| Valproic Acid | D014635 | 99-66-1 | Decreases expression |
| Vinclozolin | C025643 | 50471- | Affects expression |
| | | 44-8 | |
| Vinclozolin | C025643 | 50471- | Decreases expression |
| | | 44-8 | |

In one embodiment, the agent is Acetaminophen. In one embodiment, the agent is Clofibrate. In one embodiment, the agent is Cuprizone. In one embodiment, the agent is Cytarabine. In one embodiment, the agent is Dibutyl Phthalate. In one embodiment, the agent is Diethyl maleate. In one embodiment, the agent is Diethylnitrosamine. In one embodiment, the agent is Diuron. In one embodiment, the agent is Entinostat. In one embodiment, the agent is Fipronil. In one embodiment, the agent is Hydrogen Peroxide. In one embodiment, the agent is Leflunomide. In one embodiment, the agent is Nefazodone. In one embodiment, the agent is Pirinixic acid. In one embodiment, the agent is Tretinoin. In one embodiment, the agent is Trichostatin A. In one embodiment, the agent is Valproic Acid. In one embodiment, the agent is Vinclozolin.

In one embodiment, the agent is generally regarded as safe (GRAS).

In one embodiment, the agent has a bioavailability of not less than 50%, when administered orally.

The agents for use according to the invention may be, for example, present as salts or esters, in particular pharmaceutically acceptable salts or esters.

siRNAs, shRNAs, miRNAs and Antisense DNAs/RNAs

Expression of FAM46A may be modulated using post-transcriptional gene silencing (PTGS). Post-transcriptional gene silencing mediated by double-stranded RNA (dsRNA) is a conserved cellular defence mechanism for controlling the expression of foreign genes. It is thought that the random integration of elements such as transposons or viruses causes the expression of dsRNA which activates sequence-specific degradation of homologous single-stranded mRNA or viral genomic RNA. The silencing effect is known as RNA interference (RNAi) (Ralph et al. (2005) Nat. Medicine 11: 429-433). The mechanism of RNAi involves the processing of long dsRNAs into duplexes of about 21-25 nucleotide (nt) RNAs. These products are called small interfering or silencing RNAs (siRNAs) which are the sequence-specific mediators of mRNA degradation. In differentiated mammalian cells, dsRNA >30 bp has been found to activate the interferon response leading to shut-down of protein synthesis and non-specific mRNA degradation (Stark et al. (1998) Ann. Rev. Biochem. 67: 227-64). However, this response can be bypassed by using 21 nt siRNA duplexes (Elbashir et al. (2001) EMBO J. 20: 6877-88; Hutvagner et al. (2001) Science 293: 834-8) allowing gene function to be analysed in cultured mammalian cells.

shRNAs consist of short inverted RNA repeats separated by a small loop sequence. These are rapidly processed by the cellular machinery into 19-22 nt siRNAs, thereby suppressing the target gene expression.

Micro-RNAs (miRNAs) are small (22-25 nucleotides in length) non-coding RNAs that can effectively reduce the translation of target mRNAs by binding to their 3′ untranslated region (UTR). Micro-RNAs are a very large group of small RNAs produced naturally in organisms, at least some of which regulate the expression of target genes. Founding members of the micro-RNA family are let-7 and lin-4. The let-7 gene encodes a small, highly conserved RNA species that regulates the expression of endogenous protein-coding genes during worm development. The active RNA species is transcribed initially as an ˜70 nt precursor, which is post-transcriptionally processed into a mature ˜21 nt form. Both let-7 and lin-4 are transcribed as hairpin RNA precursors which are processed to their mature forms by Dicer enzyme.

The antisense concept is to selectively bind short, possibly modified, DNA or RNA molecules to messenger RNA in cells and prevent the synthesis of the encoded protein.

Methods for the design of siRNAs, shRNAs, miRNAs and antisense DNAs/RNAs to modulate the expression of a target protein, and methods for the delivery of these agents to a cell of interest are well known in the art. Furthermore, methods for specifically modulating (e.g. reducing) expression of a protein in a certain cell type within an organism, for example through the use of tissue-specific promoters are well known in the art.

Antibodies

The term “antibody” as used herein refers to complete antibodies or antibody fragments capable of binding to a selected target, and includes Fv, ScFv, F(ab′) and F(ab′)2, monoclonal and polyclonal antibodies, engineered antibodies including chimeric, CDR-grafted and humanised antibodies, and artificially selected antibodies produced using phage display or alternative techniques.

In addition, alternatives to classical antibodies may also be used in the invention, for example “avibodies”, “avimers”, “anticalins”, “nanobodies” and “DARPins”.

Methods for the production of antibodies are known by the skilled person. Alternatively, antibodies may be derived from commercial sources.

If polyclonal antibodies are desired, a selected mammal (e.g. mouse, rabbit, goat or horse) may be immunised. Serum from the immunised animal may be collected and treated according to known procedures. If the serum contains polyclonal antibodies to other antigens, the polyclonal antibodies may be purified by immunoaffinity chromatography. Techniques for producing and processing polyclonal antisera are known in the art.

Monoclonal antibodies directed against antigens (e.g. proteins) used in the invention can also be readily produced by the skilled person. The general methodology for making monoclonal antibodies by hybridomas is well known. Immortal antibody-producing cell lines can be created by cell fusion and also by other techniques such as direct transformation of B-lymphocytes with oncogenic DNA or transfection with Epstein-Barr virus. Panels of monoclonal antibodies produced against antigens can be screened for various properties, for example for isotype and epitope affinity.

An alternative technique involves screening phage display libraries where, for example, the phage express scFv fragments on the surface of their coat with a large variety of complementarity determining regions (CDRs). This technique is well known in the art.

Antibodies, both monoclonal and polyclonal, which are directed against antigens, are particularly useful in diagnosis, and those which are neutralising are useful in passive immunotherapy. Monoclonal antibodies in particular may be used to raise anti-idiotype antibodies. Anti-idiotype antibodies are immunoglobulins which carry an “internal image” of the antigen of the infectious agent against which protection is desired.

Techniques for raising anti-idiotype antibodies are known in the art. These anti-idiotype antibodies may also be useful for treatment, as well as for an elucidation of the immunogenic regions of antigens.

Introduction of Polypeptides and Polynucleotides into Cells

An agent for use in the invention may be, for example, a polypeptide or a polynucleotide. Polynucleotides and polypeptides may also need to be introduced into cells as part of the methods or screening assays of the invention.

Where the invention makes use of a polypeptide, the polypeptides may be administered directly to a cell (e.g. the polypeptide itself may be administered), or the polypeptides may be administered by introducing polynucleotides encoding the polypeptide into cells under conditions that allow for expression of the polypeptide in a cell of interest. Polynucleotides may be introduced into cells using vectors.

A vector is a tool that allows or facilitates the transfer of an entity from one environment to another. In accordance with the invention, and by way of example, some vectors used in recombinant nucleic acid techniques allow entities, such as a segment of nucleic acid (e.g. a heterologous DNA segment, such as a heterologous cDNA segment), to be transferred to a target cell. The vector may serve the purpose of maintaining the heterologous nucleic acid (e.g. DNA or RNA) within the cell, facilitating the replication of the vector comprising a segment of nucleic acid or facilitating the expression of the protein encoded by a segment of nucleic acid. Vectors may be non-viral or viral. Examples of vectors used in recombinant nucleic acid techniques include, but are not limited to, plasmids, chromosomes, artificial chromosomes and viruses. The vector may also be, for example, a naked nucleic acid (e.g. DNA). In its simplest form, the vector may itself be a nucleotide of interest.

The vectors used in the invention may be, for example, plasmid or virus vectors and may include a promoter for the expression of a polynucleotide and optionally a regulator of the promoter.

Vectors comprising polynucleotides used in the invention may be introduced into cells using a variety of techniques known in the art, such as transduction and transfection. Several techniques suitable for this purpose are known in the art, for example infection with recombinant viral vectors, such as retroviral, lentiviral, adenoviral, adeno-associated viral, baculoviral and herpes simplex viral vectors; direct injection of nucleic acids and biolistic transformation. Non-viral delivery systems include, but are not limited to, DNA transfection methods. Transfection includes a process using a non-viral vector to deliver a gene to a target cell.

Transfer of the polypeptide or polynucleotide may be performed by any of the methods known in the art which may physically or chemically permeabilise the cell membrane. Cell-penetrating peptides may also be used to transfer a polypeptide into a cell.

In addition, the invention may employ gene targeting protocols, for example the delivery of DNA-modifying agents.

The vector may be an expression vector. Expression vectors as described herein comprise regions of nucleic acid containing sequences capable of being transcribed. Thus, sequences encoding mRNA, tRNA and rRNA are included within this definition.

Expression vectors preferably comprise a polynucleotide for use in the invention operably linked to a control sequence that is capable of providing for the expression of the coding sequence by the host cell. A regulatory sequence “operably linked” to a coding sequence is ligated in such a way that expression of the coding sequence is achieved under conditions compatible with the control sequence. The control sequence may be modified, for example by the addition of further transcriptional regulatory elements to make the level of transcription directed by the control sequence more responsive to transcriptional modulators.

Polynucleotides

Polynucleotides of the invention may comprise DNA or RNA. They may be single-stranded or double-stranded. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

The polynucleotides may be modified by any method available in the art. Such modifications may be carried out in order to enhance the in vivo activity or lifespan of the polynucleotides of the invention.

Polynucleotides, such as DNA polynucleotides, may be produced recombinantly, synthetically or by any means available to the skilled person. They may also be cloned by standard techniques.

Longer polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA, for example mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

Proteins

The term “protein” as used herein includes single chain polypeptide molecules as well as multiple-polypeptide complexes where individual constituent polypeptides are linked by covalent or non-covalent means. The terms “polypeptide” and “peptide” as used herein refer to a polymer in which the monomers are amino acids and are joined together through peptide or disulfide bonds.

Variants, Derivatives, Analogues, Homologues and Fragments

In addition to the specific proteins and nucleotides mentioned herein, the invention also encompasses variants, derivatives, analogues, homologues and fragments thereof.

In the context of the invention, a variant of any given sequence is a sequence in which the specific sequence of residues (whether amino acid or nucleic acid residues) has been modified in such a manner that the polypeptide or polynucleotide in question retains at least one of its endogenous functions. A variant sequence can be obtained by addition, deletion, substitution, modification, replacement and/or variation of at least one residue present in the naturally occurring polypeptide or polynucleotide.

The term “derivative” as used herein in relation to proteins or polypeptides of the invention includes any substitution of, variation of, modification of, replacement of, deletion of and/or addition of one (or more) amino acid residues from or to the sequence, providing that the resultant protein or polypeptide retains at least one of its endogenous functions.

The term “analogue” as used herein in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3, to 10 or 20 substitutions, provided that the modified sequence retains the required activity or ability. Amino acid substitutions may include the use of non-naturally occurring analogues.

Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The term “homologue” as used herein means an entity having a certain homology with the wild type amino acid sequence or the wild type nucleotide sequence. The term “homology” can be equated with “identity”.

In the present context, a homologous sequence is taken to include an amino acid sequence which may be at least 50%, 55%, 65%, 75%, 85% or 90% identical, preferably at least 95% or 97% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence which may be at least 50%, 55%, 65%, 75%, 85% or 90% identical, preferably at least 95% or 97% or 99% identical to the subject sequence. Although homology can also be considered in terms of similarity, in the context of the present invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Homology comparisons can be conducted by eye, or more usually, with the aid of readily available sequence comparison programs. These commercially available computer programs can calculate percent homology or identity between two or more sequences.

Percent homology may be calculated over contiguous sequences, i.e. one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the amino acid or nucleotide sequence may cause the following residues or codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids or nucleotides, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestf it package the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestf it package (University of Wisconsin, USA; Devereux et al. (1984) Nucleic Acids Research 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, BLAST 2 Sequences, is also available for comparing protein and nucleotide sequences (FEMS Microbiol. Lett. (1999) 174(2):247-50; FEMS Microbiol. Lett. (1999) 177(1):187-8).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix (the default matrix for the BLAST suite of programs). GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Codon Optimisation

The polynucleotides used in the invention may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. Codon usage tables are known in the art for mammalian cells, as well as for a variety of other organisms.

Method of Treatment

All references herein to treatment include curative, palliative and prophylactic treatment. The treatment of mammals, particularly humans, is preferred. Both human and veterinary treatments are within the scope of the invention.

Administration

Although the agents for use in the invention can be administered alone, they will generally be administered in admixture with a pharmaceutical carrier, excipient or diluent, particularly for human therapy.

In some embodiments, the agent is a nutritional agent, food additive or food ingredient, and may thus be formulated in a suitable food composition. Thus, the agent may be administered, for example, in the form of a food product, drink, food supplement, nutraceutical, nutritional formula or pet food product.

Dosage

The skilled person can readily determine an appropriate dose of an agent of the invention to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. There can of course be individual instances where higher or lower dosage ranges are merited, and such are within the scope of the invention.

Subject

The term “subject” as used herein refers to either a human or non-human animal.

Examples of non-human animals include vertebrates, for example mammals, such as non-human primates (particularly higher primates), dogs, rodents (e.g. mice, rats or guinea pigs), pigs and cats. The non-human animal may be a companion animal.

Preferably, the subject is a human.

The skilled person will understand that they can combine all features of the invention disclosed herein without departing from the scope of the invention as disclosed.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

This study relates to a protein quantitative trait loci (pQTL) and expression quantitative trait loci (eQTL) analyses performed on Diogenes weight loss intervention data. The Diogenes study is a pan-European, randomised and controlled dietary intervention study investigating the effects of dietary protein and glycaemic index on weight loss and weight maintenance in obese and overweight families in eight European centres (Larsen et al. (2009) Obesity Rev. 11: 76-91). In brief, the Diogenes study subjected screened participants to a low-calorie diet (LCD) phase (CID1), in which the overweight/obese subjects followed an 8 week ModifastR diet (approximately 800 kCal/day), followed by a weight maintenance phase (CID2).

This is the first study that has tested the association between common variants genotyped on an Illumina chip and protein expression change during intervention focusing on proteins associated to body mass index (BMI) change.

### EXAMPLES

### Example 1

pQTL Effects on Leptin Protein Expression

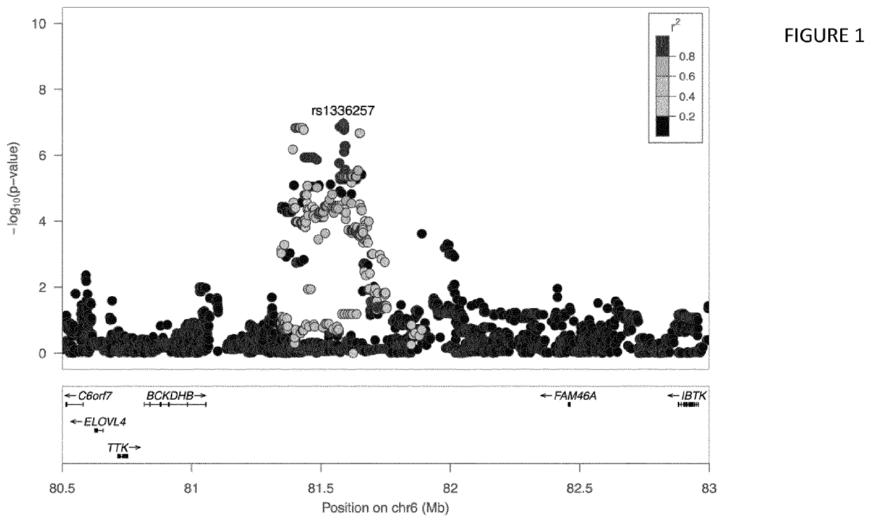

Among the 42 proteins associated with BMI both at baseline and during the LCD, only leptin displayed a significant pQTL signal during LCD. Two tightly linked SNPs in an intergenic region on chromosome 6 were associated with leptin protein expression changes (FIG. 1). Surrounding genes included BCKDHB, involved in the catabolism of branched chain amino acids (BCAAs); and FAM46A, a SMAD signalling pathway related protein involved in TGF-δ signaling pathway. Using adipocyte expression data from their study, the inventors performed an eQTL analysis. Only the FAM46A gene expression displayed association with SNPs in the pQTL region. No other gene within 5 Mb around the pQTL locus displayed eQTL association signals higher than the FAM46A signal in their data. The biological role of FAM46A is largely unknown. The protein interacts with the BAG6 protein involved in gene regulation, apoptosis, regulation of protein synthesis and degradation. It has recently been shown that BAG6 splicing in subcutaneous fat is highly determined by BMI. BAG6 is also involved in stress response. There is a reciprocal relationship between heat shock protein HSP70 and BAG6 suggesting that BAG6 could be a central regulator of the cellular content of HSP70 which is in turn positively associated with leptin in type 2 diabetes and in their data (p=3.4×10−4 from a multivariate regression adjusted for age, gender, center and BMI change during LCD intervention). pQTL association signals in the regulatory region of FAM46A were also observed for HSP70 (data not shown). Adjusting for leptin expression change as a confounding cofactor did not affect pQTL results (data not shown). In other words, the HSP70 pQTL signal in the FAM46A regulatory region was leptin-level independent.

### Example 2

Relationship Between FAM46A and Leptin

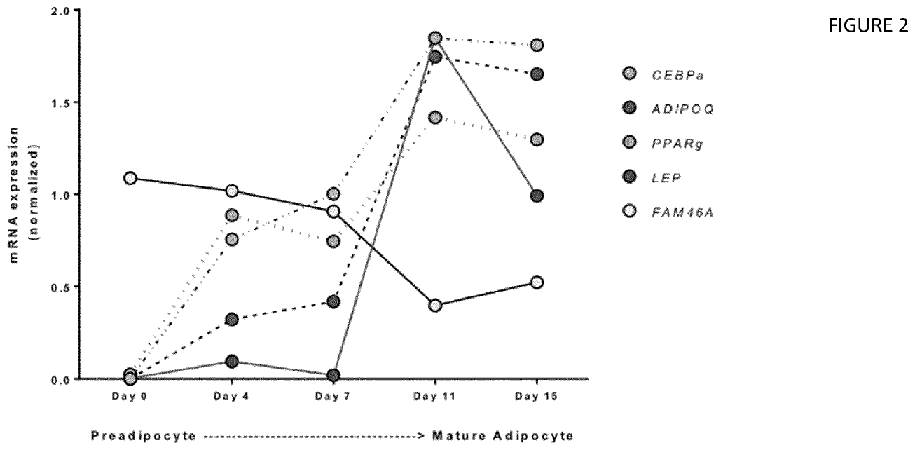

The reciprocal relationship between FAM46A and leptin is also apparent during adipocyte maturation. In pre-adipocytes FAM46A expression is high and decreases during maturation to a minimum at day seven. This decrease coincides with the appearance of leptin (FIG. 2). To complete the genetic characterization of the QTL results we evaluated the directionality of pQTL and eQTL SNPs. The inventors first focused on the 2 pQTL SNPs significantly associated with leptin change during LCD. SNP rs9344031 was positively associated with leptin protein level change (p=1.48×10−7) and negatively associated with FAM46A gene expression change (p=4.9×10−3). The same trend was found for the second pQTL SNP rs481777 although gene expression did not reach nominal significance (p=0.12) likely because of the difference in sample size between the eQTL and pQTL study. In an enlarged analysis, pQTL signals were extracted in the promoter region of FAM46A located between 81.3 and 81.8 Mb on chromosome 6 (FIG. 1) for all genotyped and imputed SNPs. Of 1,833 SNPs in this region, 94 were identified as pQTLs and eQTLs for leptin and FAM46A, respectively, assuming an uncorrected 5% nominal significance level. All QTL SNPs displayed opposite association direction between leptin protein and FAM46A gene expression, consistent with a regulatory role of FAM46A on leptin.

### Example 3

Knockdown of FAM46A

Silencing of the FAM46A gene was achieved by RNA interference through electroporation. Briefly, on day 7 of differentiation, human adipocyte SGBS cells were detached with trypsin/EDTA and counted. Silencer Select Negative Control siRNA (siNeg) or gene-specific siRNA targeting human FAM46A (Life Technologies, Carlsbad, USA) were delivered into adipocytes at a concentration of 25 nM per 400,000 cells with the Neon Transfection System (Invitrogen, Madison, Wis., USA) using the following parameters: 1,100 V, 20 ms, 1 pulse. After transfection cells were incubated for 48 hr in the same media.

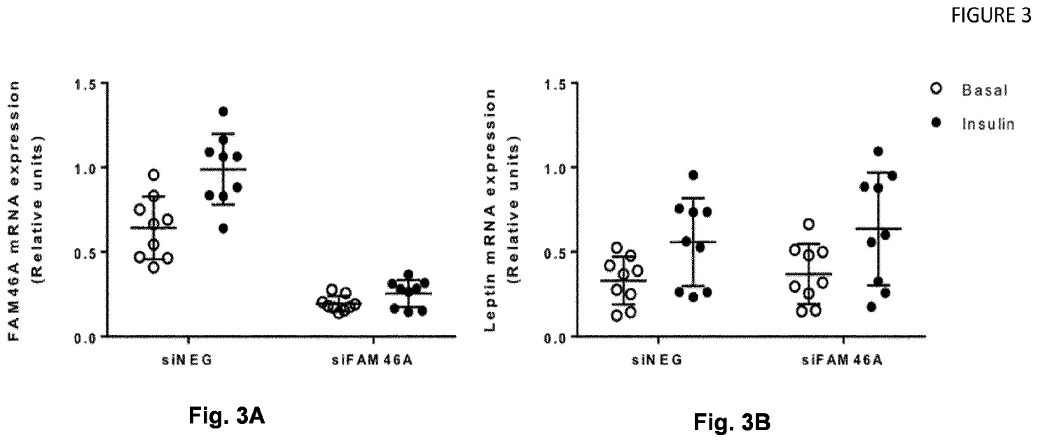

Transfection with siRNA resulted in a 70% and 74% reduction of FAM46A gene expression in the basal and insulin stimulated state, respectively. FAM46A protein was reduced by 31% and 73% in the basal and insulin-induced state, respectively (FIG. 3).

FAM46A knockdown had no influence on leptin gene expression and did not show any effect on markers of adipocyte differentiation PPARγ and CEBPα (data not shown).

Conversely, FAM46A knockdown resulted in a highly significant 49% increase of insulin stimulated leptin secretion from the adipocytes (p=0.0002 one-sided t test for leptin increase). Under conditions without prior insulin stimulation the knockdown still showed a marginal 19% increase in leptin secretion (data not shown). To evaluate whether the effects of the knockdown of FAM46A were specific for leptin, the secretion of adiponectin was tested. Secretion of adiponectin was not affected by the knockdown (data not shown).

### Example 4

Overexpression of FAM46A

Overexpression of the FAM46A gene was achieved by cDNA transfection through electroporation. Briefly, on day 7 of differentiation, SGBS cells were detached with trypsin/EDTA and counted. pCMV6 empty vector (Control) or FAM46A (NM_017633) full human cDNA ORF Clone (OriGene, Rockville, Md. 20850, USA) were delivered into adipocytes at a concentration of 0.5 μg per 400,000 cells with the Neon Transfection System (Invitrogen, Madison, Wis., USA) using the following parameters: 1,100 V, 20 ms, 1 pulse. After transfection cells were incubated for 48 hr in the same media.

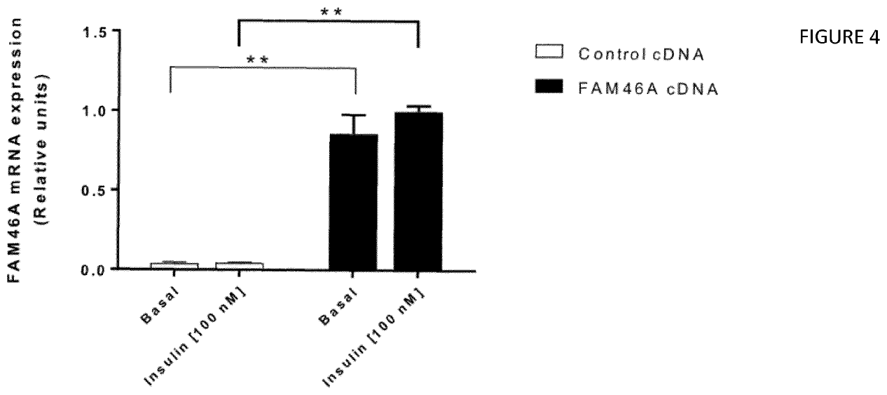

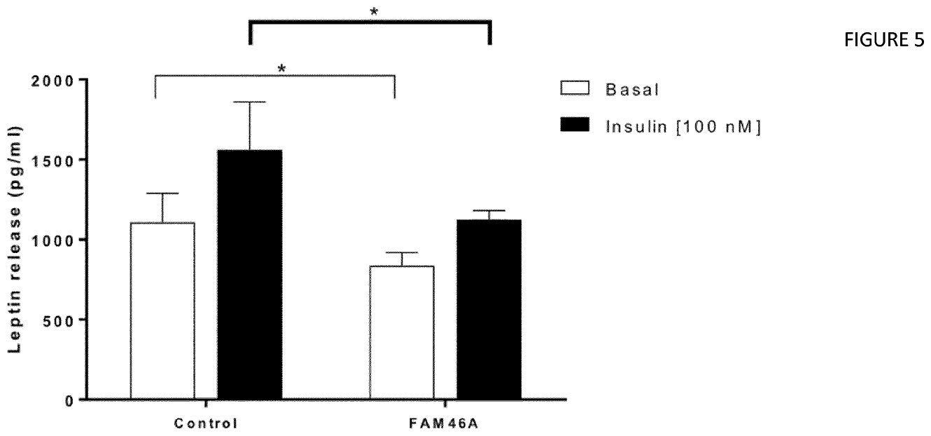

Transfection of SGBS cells resulted in a 21-fold increase of FAM46A expression (FIG. 4). FAM46A overexpression resulted in a significant 28% and 24% (p=0.034 and 0.038, one-sided t test, for both basal and insulin stimulated states, respectively) reduction of insulin and non-insulin stimulated leptin release, respectively (FIG. 5). Overexpression did not influence PPARγ or CEBPα gene expression (data not shown). Adiponectin release was not affected by FAM46A overexpression, both in the basal as well as the insulin stimulated state (data not shown).

In order to measure gene expression for FAM46A, leptin and PPARγ, total RNA from SGBS adipocytes was isolated using the Rneasy Plus Mini kit (Qiagen) at day nine. After assessing quantity and quality with a Nano-Drop, 0.5 μg total RNA was reverse transcribed using the High Capacity cDNA Reverse Transcription kit (Applied Biosystems, Foster City, Calif., USA) according to manufacturer's instructions.

Expression levels of mRNA were determined using a SYBR Green kit LightCycler 1536 DNA Green Master kit using the LightCycler Instrument (Roche, Basel, Switzerland). Oligo sequences were designed using the Biology Workbench website. Human primers were obtained from Life Technologies Europe. FAM46A, Leptin, CEBPα and PPARγ mRNA levels were normalized against TATA box binding protein (TBP). Primers used as listed in the table below.

Primer sequences. Forward and reverse primer sequences.

| GENE NAME | PRIMER SEQUENCE |
|---|---|
| TBP | Forward primer-TGGTGTGCACAGGAGCCAAG |
| (Housekeeping gene) | (SEQ ID NO: 7) |
| | Reverse primer-TTCACATCACAGCTCCCCAC |
| | (SEQ ID NO: 8) |
| LEP | Forward primer-GGCTTTGGCCCTATCTTTTC |
| | (SEQ ID NO: 9) |
| | Reverse primer-ACCGGTGACTTTCTGTTTGG |
| | (SEQ ID NO: 10) |
| FAM46A | Forward primer-CAACAGTGGCAAAAATGTGG |
| | (SEQ ID NO: 11) |
| | Reverse primer-TCCTGGAAATCGCCATAGAC |
| | (SEQ ID NO: 12) |
| PPARγ | Forward primer- |
| | GATCCAGTGGTTGCAGATTACAA (SEQ ID NO: 13) |
| | Reverse primer-GAGGGAGTTGGAAGGCTCTTC |
| | (SEQ ID NO: 14) |
| CEBPα | Forward primer-TGGACAAGAACAGCAACGAG |
| | (SEQ ID NO: 15) |
| | Reverse primer-TTGTCACTGGTCAGCTCCAG |
| | (SEQ ID NO: 16) |
| ADIPOQ | Forward primer-GGCCGTGATGGCAGAGAT |
| | (SEQ ID NO: 17) |
| | Reverse primer-CCTTCAGCCCGGGTACT |
| | (SEQ ID NO: 18) |

All publications mentioned in the above specification are herein incorporated by reference. Various modifications and variations of the disclosed agents, uses and methods of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A method of supporting weight maintenance and/or treating or preventing obesity, the method comprising administering an agent capable of decreasing the activity of FAM46A to an individual in need thereof,
wherein the agent is selected from the agents listed in Table 1.

2. Method according to claim 1, wherein the agent is administered to a subject during or after a weight loss intervention.

3. Method according to claim 1, wherein the agent increases leptin levels in a subject.

4. Method according to claim 1, wherein the agent increases leptin levels in a subject.

5. Method according to claim 1, wherein the agent decreases the level of FAM46A in a subject.

6. Method according to claim 1, wherein the agent is selected from the group consisting of acetaminophen, clofibrate, cuprizone, cytarabine, dibutyl phthalate, diethyl maleate, diethylnitrosamine, diuron, entinostat, fipronil, hydrogen peroxide, leflunomide, nefazodone and pirinixic acid.

7. (canceled)

8. A method of identifying an agent capable of supporting weight maintenance and/or treating or preventing obesity in a subject comprising the steps:
(a) contacting a preparation comprising a FAM46A polypeptide or polynucleotide with a candidate agent; and
(b) detecting whether the candidate agent affects the activity of the FAM46A polypeptide or polynucleotide.

9. A method of identifying an agent that decreases the activity of FAM46A comprising the steps:
(a) contacting a preparation comprising a FAM46A polypeptide or polynucleotide with a candidate agent; and
(b) detecting whether the candidate agent affects the activity of the FAM46A polypeptide or polynucleotide.

10. The method of claim 8, wherein the preparation comprising the FAM46A polypeptide or polynucleotide comprises a cell comprising the FAM46A polypeptide or polynucleotide.

11. The method of claim 10, wherein the cell is an adipocyte.

12. The method of claim 8, wherein the method is for identifying an agent that decreases the expression of FAM46A.

13. The method of claim 8, wherein the method further comprises detecting whether the candidate agent affects the levels of leptin produced by the cell.

14. The method of claim 8, wherein the candidate agent is a natural product.

15. The method of claim 9, wherein the preparation comprising the FAM46A polypeptide or polynucleotide comprises a cell comprising the FAM46A polypeptide or polynucleotide.

16. The method of claim 15, wherein the cell is an adipocyte.

17. The method of claim 9, wherein the method is for identifying an agent that decreases the expression of FAM46A.

18. The method of claim 9, wherein the method further comprises detecting whether the candidate agent affects the levels of leptin produced by the cell.

19. The method of claim 9, wherein the candidate agent is a natural product.


SEQUENCE LISTING (18 sequences)

SEQ ID NO: 1 - protein, 442 aa, Homo sapiens
  FAM46 prot SEQ ID NO 1
MAEGEGYFAMSEDELACSPYIPLGGDFGGGDFGGGDFGGGDFGGGGSFGGHCLDYCESPTAHCNVLNWEQVQRLDGILSETIPIHGRGNFPTLELQPSLIVKVVRRRLAEKRIGVRDVRLNGSAASHVLHQDSGLGYKDLDLIFCADLRGEGEFQTVKDVVLDCLLDFLPEGVNKEKITPLTLKEAYVQKMVKVCNDSDRWSLISLSNNSGKNVELKFVDSLRRQFEFSVDSFQIKLDSLLLFYECSENPMTETFHPTIIGESVYGDFQEAFDHLCNKIIATRNPEEIRGGGLLKYCNLLVRGFRPASDEIKTLQRYMCSRFFIDFSDIGEQQRKLESYLQNHFVGLEDRKYEYLMTLHGVVNESTVCLMGHERRQTLNLITMLAIRVLADQNVIPNVANVTCYYQPAPYVADANFSNYYIAQVQPVFTCQQQTYSTWLPCN

SEQ ID NO: 2 - DNA, 1329 bp, Homo sapiens
  source 1..1329: /mol_type=“unassigned DNA” /note=“FAM46 DNA SEQ ID NO 2” /organism=“Homo sapiens”
atggcggagggtgaagggtacttcgccatgtctgaggacgagctggcctgcagcccctacatccccctaggcggcgacttcggcggcggcgacttcggcggcggcgacttcggcggcggcgacttcggcggtggcggcagcttcggtgggcattgcttggactattgcgaaagccctacggcgcactgcaatgtgctgaactgggagcaagtgcagcggctggacggcatcctgagcgagaccattccgattcacgggcgcggcaacttccccacgctcgagctgcagccgagcctgatcgtgaaggtggtgcggcggcgcctggccgagaagcgcattggcgtccgcgacgtgcgcctcaacggctcggcagccagccatgtcctgcaccaggacagcggcctgggctacaaggacctggacctcatcttctgcgccgacctgcgcggggaaggggagtttcagactgtgaaggacgtcgtgctggactgcctgttggacttcttacccgagggggtgaacaaagagaagatcacaccactcacgctcaaggaagcttatgtgcagaaaatggttaaagtgtgcaatgactctgaccgatggagtcttatatccctgtcaaacaacagtggcaaaaatgtggaactgaaatttgtggattccctccggaggcagtttgaattcagtgtagattcttttcaaatcaaattagactctcttctgctcttttatgaatgttcagagaacccaatgactgagacatttcaccccacaataatcggggagagcgtctatggcgatttccaggaagcctttgatcacctttgtaacaagatcattgccaccaggaacccagaggaaatccgagggggaggcctgcttaagtactgcaacctcttggtgaggggctttaggcccgcctctgatgaaatcaagacccttcaaaggtatatgtgttccaggtttttcatcgacttctcagacattggagagcagcagagaaaactggagtcctatttgcagaaccactttgtgggattggaagaccgcaagtatgagtatctcatgacccttcatggagtggtaaatgagagcacagtgtgcctgatgggacatgaaagaagacagactttaaaccttatcaccatgctggctatccgggtgttagctgaccaaaatgtcattcctaatgtggctaatgtcacttgctattaccagccagccccctatgtagcagatgccaactttagcaattactacattgcacaggttcagccagtattcacgtgccagcaacagacctactccacttggctaccctgcaattaa

SEQ ID NO: 3 - protein, 447 aa, Homo sapiens
  FAM46 AAH07351.1 SEQ ID NO 3
MAEGEGYFAMSEDELACSPYIPLGGDFGGGDFGGGDFGGGDFGGGDFGGGGSFGGHCLDYCESPTAHCNVLNWEQVQRLDGILSETIPIHGRGNFPTLELQPSLIVKVVRRRLAEKRIGVRDVRLNGSAASHVLHQDSGLGYKDLDLIFCADLRGEGEFQTVKDVVLDCLLDFLPEGVNKEKITPLTLKEAYVQKMVKVCNDSDRWSLISLSNNSGKNVELKFVDSLRRQFEFSVDSFQIKLDSLLLFYECSENPMTETFHPTIIGESVYGDFQEAFDHLCNKIIATRNPEEIRGGGLLKYCNLLVRGFRPASDEIKTLQRYMCSRFFIDFSDIGEQQRKLESYLQNHFVGLEDRKYEYLMTLHGVVNESTVCLMGHERRQTLNLITMLAIRVLADQNVIPNVANVTCYYQPAPYVADANFSNYYIAQVQPVFTCQQQTYSTWLPCN

SEQ ID NO: 4 - DNA, 1344 bp, Homo sapiens
  source 1..1344: /mol_type=“unassigned DNA” /note=“FAM46A BC007351.2 SEQ ID NO4” /organism=“Homo sapiens”
atggcggagggtgaagggtacttcgccatgtctgaggacgagctggcctgcagcccctacatccccctaggcggcgacttcggcggcggcgacttcggcggcggcgacttcggcggcggcgacttcggcggcggcgacttcggcggtggcggcagcttcggtgggcattgcttggactattgcgaaagccctacggcgcactgcaatgtgctgaactgggagcaagtgcagcggctggacggcatcctgagcgagaccattccgattcacgggcgcggcaacttccccacgctcgagctgcagccgagcctgatcgtgaaggtggtgcggcggcgcctggccgagaagcgcattggcgtccgcgacgtgcgcctcaacggctcggcagccagccatgtcctgcaccaggacagcggcctgggctacaaggacctggacctcatcttctgcgccgacctgcgcggggaaggggagtttcagactgtgaaggacgtcgtgctggactgcctgttggacttcttacccgagggggtgaacaaagagaagatcacaccactcacgctcaaggaagcttatgtgcagaaaatggttaaagtgtgcaatgactctgaccgatggagtcttatatccctgtcaaacaacagtggcaaaaatgtggaactgaaatttgtggattccctccggaggcagtttgaattcagtgtagattcttttcaaatcaaattagactctcttctgctcttttatgaatgttcagagaacccaatgactgagacatttcaccccacaataatcggggagagcgtctatggcgatttccaggaagcctttgatcacctttgtaacaagatcattgccaccaggaacccagaggaaatccgagggggaggcctgcttaagtactgcaacctcttggtgaggggctttaggcccgcctctgatgaaatcaagacccttcaaaggtatatgtgttccaggtttttcatcgacttctcagacattggagagcagcagagaaaactggagtcctatttgcagaaccactttgtgggattggaagaccgcaagtatgagtatctcatgacccttcatggagtggtaaatgagagcacagtgtgcctgatgggacatgaaagaagacagactttaaaccttatcaccatgctggctatccgggtgttagctgaccaaaatgtcattcctaatgtggctaatgtcacttgctattaccagccagccccctatgtagcagatgccaactttagcaattactacattgcacaggttcagccagtattcacgtgccagcaacagacctactccacttggctaccctgcaattaa

SEQ ID NO: 5 - protein, 167 aa, Homo sapiens
  Leptin NP_000221.1 SEQ ID NO5
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 6 - DNA, 504 bp, Homo sapiens
  source 1..504: /mol_type=“unassigned DNA” /note=“Leptin NM_000230.2 SEQ ID NO6” /organism=“Homo sapiens”
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgctga

SEQ ID NO: 7 - DNA, 20 bp, Homo sapiens
  source 1..20: /mol_type=“unassigned DNA” /organism=“Homo sapiens”
tggtgtgcacaggagccaag

SEQ ID NO: 8 - DNA, 20 bp, Homo sapiens
  source 1..20: /mol_type=“unassigned DNA” /organism=“Homo sapiens”
ttcacatcacagctccccac

SEQ ID NO: 9 - DNA, 20 bp, Homo sapiens
  source 1..20: /mol_type=“unassigned DNA” /organism=“Homo sapiens”
ggctttggccctatcttttc

SEQ ID NO: 10 - DNA, 20 bp, Homo sapiens
  source 1..20: /mol_type=“unassigned DNA” /organism=“Homo sapiens”
accggtgactttctgtttgg

SEQ ID NO: 11 - DNA, 20 bp, Homo sapiens
  source 1..20: /mol_type=“unassigned DNA” /organism=“Homo sapiens”
caacagtggcaaaaatgtgg

SEQ ID NO: 12 - DNA, 20 bp, Homo sapiens
  source 1..20: /mol_type=“unassigned DNA” /organism=“Homo sapiens”
tcctggaaatcgccatagac

SEQ ID NO: 13 - DNA, 23 bp, Homo sapiens
  source 1..23: /mol_type=“unassigned DNA” /organism=“Homo sapiens”
gatccagtggttgcagattacaa

SEQ ID NO: 14 - DNA, 21 bp, Homo sapiens
  source 1..21: /mol_type=“unassigned DNA” /organism=“Homo sapiens”
gagggagttggaaggctcttc

SEQ ID NO: 15 - DNA, 20 bp, Homo sapiens
  source 1..20: /mol_type=“unassigned DNA” /organism=“Homo sapiens”
tggacaagaacagcaacgag

SEQ ID NO: 16 - DNA, 20 bp, Homo sapiens
  source 1..20: /mol_type=“unassigned DNA” /organism=“Homo sapiens”
ttgtcactggtcagctccag

SEQ ID NO: 17 - DNA, 18 bp, Homo sapiens
  source 1..18: /mol_type=“unassigned DNA” /organism=“Homo sapiens”
ggccgtgatggcagagat

SEQ ID NO: 18 - DNA, 17 bp, Homo sapiens
  source 1..17: /mol_type=“unassigned DNA” /organism=“Homo sapiens”
ccttcagcccgggtact